(19) United States
(12) Patent Application Publication
(10) Pub. No.: US 2009/0252646 A1
(43) Pub. Date: Oct. 8, 2009

(54) STERILIZATION METHODS AND SYSTEMS FOR GAMING EQUIPMENT

(75) Inventors: H. Lee HOLDEN, Los Gatos, CA (US); Josh P. DEFOSSET, Boulder Creek, CA (US)

Correspondence Address: LEVINE BAGADE HAN LLP, 2400 GENG ROAD, SUITE 120, PALO ALTO, CA, 94303, US

(73) Assignee: Invention Factory, LLC, Los Gatos, CA (US)

(21) Appl. No.: 12/474,138

(22) Filed: May 28, 2009

Related U.S. Application Data
(63) Continuation of application No. PCT/US2008/050503, filed on Jan. 8, 2008.
(60) Provisional application No. 60/883,944, filed on Jan. 8, 2007.
(60) Provisional application No. 60/885,829, filed on Jan. 19, 2007.

(51) Int. Cl. A61L 2/02; B01J 19/12; B01J 19/00; A63F 13/00; A63F 11/00

(52) U.S. Cl. 422/24; 422/186.3; 422/129; 463/30; 273/274

(57) ABSTRACT

Enclosures that include light generating sterilization sources and conveying systems for sterilizing gaming items are disclosed. Also disclosed are gaming equipment or tables having user interface portions coated or treated with a photo-catalyst for maintaining sterility of the user interface.

### CROSS-REFERENCE TO RELATED APPLICATIONS

This application is a continuation of International Application No. PCT/US2008/050503, filed Jan. 8, 2008, which claims priority to U.S. Provisional Application No. 60/883,944, filed Jan. 8, 2007 and U.S. Provisional Application No. 60/885,829, filed Jan. 19, 2007, the contents of which are incorporated herein by reference in their entireties.

### BACKGROUND OF THE INVENTION

The popularity of air sterilizers, anti-bacterial soaps and the many new UV and Ozone sterilized household products appears to indicate a consumer's desire to use sanitized products. Today consumers have available: UV Toothbrush, as well as a companion UV toothbrush travel kits, a UV Wand that sterilizes a glass of drinking water, a computer mouse from IO Gear with a photocatalyst self-sanitizing top surface and the Ozone producing systems such as the new Lotus Sanitizing System that breaks down pesticides and bacteria on fruit and vegetables. UV light sterilization is a safe and proven technology that is widely used in hospitals, air and water purification, food processing and packaging, and medical device and packaging sterilization. UV light sterilization is also found in toothbrush holders and surface cleaning sterilizers.

Given the trend of media reports on E-Coli, avian flu (H5N1 virus), anthrax, SARS (Coronavirus), and other contagious diseases consumers are becoming more and more health conscious and cognizant of the danger of exposing themselves and loved ones to germs, bacteria, viruses and other dangerous pathogens. Employers are also becoming more and more aware of the losses incurred by the spread between employees of contagious diseases that are relatively mild in nature.

Accordingly, the likelihood of passing germs, bacteria, viruses or other dangerous pathogens is problematic in forums where items are passed as a matter of course between the public and employees. The problem increases when the forum includes fixed equipment that is shared by individuals. For example, casino chips, dice, cards, cash or other items pass between countless individuals in casinos. Moreover, in casinos countless individuals use the same slot machines, video poker machines, or other electronic games of chance. In many cases, these items are not sterilized when delivered to the public. In any event, it is impractical for the casino operator to sterilize these items on a routine basis. Also, all Hotels have courtesy phones, elevator enclosures and elevator buttons that are impractical to clean after use by each individual patron. Finally, ATM machines, in high-traffic locations such as banks or gas stations, as well as other point-of-sale devices located in retailers that encourage the customer to use the device themselves, are all candidates for this method of sanitization

While this problem affects any public forum where items are passed between the attendees or where individuals share use of equipment, casinos, unlike other public forums, are particularly susceptible to the transmission of such germs because of the casino environment that usually consists of crowds of people, from all corners of the globe, gambling, socializing and drinking & smoking (both actions transferring pathogens hand to mouth) for many hours a day.

Accordingly, a need remains for cost effective solution to reduce the transmission of pathogens between individuals or even employees of the forum.

Certain compounds, when exposed to light, set in motion a particular chemical reaction. These compounds are called photo-catalysts. Some photo-catalysts may render the UV sterilization more effective. One such photocatalyst includes Titanium Dioxide (TiO2), also known as titania. Titania is one of the earth's most abundant resources and is extracted from beach sands. Titania has been used for decades in the rutile physical state (rutile crystalline structure) as an opacifier in paint (makes the paint not be translucent or “see-through” like a stain). Recently, many advances have been made in the anatase physical state (anatase crystalline structure) of titania. Unlike the rutile form, the anatase form acts as a photo-catalyst. The chemical definition of a catalyst is a compound that sets in motion a chemical reaction without actually being consumed by the reaction (if it was consumed, it would be a reagent).

In the case of titania, there are two reactions that are ignited by exposure to short wavelength light (380 nm is the apex of this reaction). First, the titania breaks down water vapor in the surrounding air. The water molecule splits into OH— and O2+ (Hydroxyl radical and superoxide anion). These short-lived radicals are extremely aggressive and break down viruses, bacteria, mold, fungi and other pathogens. It is worth noting that these radicals are so short-lived that the sterilization effect is only active on the surface actually coated with titania and exposed to light. Second, titania will also initiate a reaction with any nearby volatile organic compounds (VOCs) breaking them down in a cascade reaction until they are extremely short molecules like CO2, O2 and minute amounts of free hydrogen (H2). While this second reaction offers many other uses for titania, it will provide for us an air cleaning effect (Smokey and other odors will be locally reduced), but, it is the first reaction with water vapor that provides the sterilization effects of titania.

As stated above, the range of the titanium dioxide reaction is 350-388 nm and the greatest efficacy is achieved at 380 nm. Many light sources will provide some of their output at or near the 380 nm wavelength, including: UV-A lamps (best), sunlight, fluorescent light fixtures (cold Cathode or traditional), and, to a limited degree UV-C producing bulbs. One means of sterilizing contemplated by this disclosure is to activate the titanium dioxide with any light source with some output in the 380 nm wavelength. Although, using a UV-C bulb is the least efficient of the 4 means above, it will work. Virtually all locations suitable for the sterilization means discussed herein are already either lit by daylight or by fluorescent lights. So, one desired embodiment is to use either existing lighting, or, if we must add additional lighting, it will be UV-A as it offers the greatest amount of 380 nm light. UV-A is not in itself germicidal. However, UV-A combined with a photocatalyst will effectively reduce the amount of pathogens on the user interface.

Ultra-Violet (UV) light sterilization is a safe and proven technology. It is widely used in hospitals, air and water purification, food processing and packaging, medical packaging, and increasingly, in home products such as toothbrush and surface sterilizers.

As shown by the Chart in FIG. 1A, UV light is part of the light spectrum between 100 and 400 nanometers (nm), just below the violet end of the visible spectrum. UV technology is a non-chemical approach to disinfection. In this method of disinfection, no chemicals are added, which makes this process simple, inexpensive and requires very low maintenance. It should be noted that there are only two other non-chemical approaches to sterilization recognized by the FDA and USDA: steam and gamma radiation, both of which are hazardous and impractical for home use. UV sterilizers utilize germicidal lamps that are designed and calculated to produce a certain dosage of Ultra-Violet light. The principle of design is based on a product of time and intensity—to ensure success, the system must provide sufficient levels of both time and intensity for proper disinfection.

UV light rays with wavelengths shorter than 300 nm are extremely effective in killing microorganisms. The most effective sterilizing range for UV is within the C bandwidth (UVC=200 to 280 nm), and the apex of this range is generally considered to be 253.7 nm. This range is called the germicidal bandwidth. FIG. 1B represents the germicidal ultraviolet light killing cells by damaging the DNA of the cells. The light initiates a reaction between two molecules of thymine, one of the bases that makes up DNA. UV light at the UVC wavelength causes adjacent thymine molecules on the DNA strand to dimerize. The resulting thymine dimmer is very stable. If a sufficient amount of defects accumulate on a microorganism's DNA, the organism is unable to replicate and is rendered harmless. UVC has been used in hospitals for decades to sterilize surgical instruments, water (including IV fluids), and the air in operating rooms. Many food and drug companies use germicidal lamps to disinfect various types of medical devices and packaging as well as food packaging.

Accordingly, there remains to be a need for improved systems and methods to sanitize or sterilize such gaming supplies or equipment in a rapid manner and in sufficient quantities without disrupting the operation of the casino or other gaming forum. While the principles described below are shown in applications in the gaming industry, the applications and uses of the inventions are clearly applicable in other forums where a relatively large number of items are passed among groups of people or where individuals use equipment, machines or other apparatus having a user interface. Some such additional examples include, electronic racetrack wagering equipment, automatic teller machines, interactive information directories, shared telephones, shared computer terminals, etc.

### SUMMARY OF THE INVENTION

The devices and methods described herein are improved means for sterilizing gaming items as well as providing self-sterilizing gaming tables and machines.

In one variation, the invention includes a sterilizing unit for sterilizing gaming items comprising a housing, a light generating sterilizing source, a conveyor system having at least a first conveyor portion and a second conveyor portion, each conveyor portion placed such that a plurality of surfaces of the gaming item are directly exposed to the light generating sterilizing source, and where the first conveyor portion includes a first plurality of seats for nesting of at least one of the gaming items, and where the second conveyor portion includes a second plurality of seats for nesting at least one of the gaming items, where the first and second conveyor portions are placed relative to one another such that movement of the gaming item from the first conveyor portion to the second conveyor portion causes the gaming item to change orientation when nested in the second conveyor portion.

As noted herein, the light generating sterilizing source can include a UV illumination source coupled to a power supply such that the light source is configured to generate light between 100 and 400 nanometers wavelength (either UV-A, UV-B, and/or UV-C). The source may comprise a conventional bulb, filament, or LED. The various types of sterilizing modalities discussed herein can include sterilizing means.

In another variation, the invention includes a method for providing a gaming item sterilization system, the method comprising, creating a sterilization path having a light-based sterilization source within an enclosure, advancing the gaming item on a conveyor through a portion of the sterilization path, such that at a plurality of surfaces of the gaming item are directly exposed to the sterilization source advancement through the sterilization path, and directly exposing each surface of the gaming item to the light-based sterilization source for a sufficient time to significantly sterilize all surfaces of the gaming item without damaging the gaming item.

In an additional variation, the above method includes automatically re-orienting the gaming item so that all surfaces of the gaming item are exposed to the light-based sterilization source.

Additional variations of the invention include gaming machines, tables, or fixtures that allow for self-sterilizing of the machine, table, or fixture.

In one such variation, the invention includes self-sterilizing casino gaming machine for engaging a user in a casino game, the machine comprising a display portion for visually displaying the game, a user interface comprising one or more portions allowing the user to engage in the game and affect the display portion, where at least a portion of the user interface comprises a photo-catalyst material that when irradiated with UV light, sterilizes the portion of the user interface.

In an additional variation, the invention includes a self-sterilizing casino gaming table comprising a playing surface having at least a covering portion containing visual marks of a casino game, and a photo-catalyst material located on at least a surface of the covering portion without obscuring the visual marks of the casino game, where when irradiated with UV light, the photo-catalyst material sterilizes the covering portion.

The visual marks of a casino game are typically the markings on the covering that denote the particular game. For example, craps, blackjack, poker, or other card games, roulette, dice, etc.

The machines, tables, or fixtures can be sterilized with ambient UV light. Alternatively, the machines, tables, or fixtures can be sterilized with an additional light source (such as a wand that illuminates the various appliances during a standard cleaning). Alternatively, the light source can be affixed to portions of the appliance for automatic sterilization.

### BRIEF DESCRIPTIONS OF THE DRAWINGS

FIG. 1A provides a chart showing UV light as part of the light spectrum between 100 and 400 nanometers (nm), just below the violet end or the visible spectrum.

FIG. 1B represents the germicidal ultraviolet light killing cells by damaging the DNA of the cells.

FIGS. 2A and 2B show a variation of systems for sanitizing gaming items in combination with conveyor systems.

FIGS. 3A and 3B show another variation of systems for sanitizing gaming items in combination with conveyor systems.

FIG. 3C illustrates an example of the conveyor having a shape or feature to accommodate the gaming item.

FIG. 4 shows another variation of the sterilization system used for playing cards.

FIG. 5 shows a variation of a system using conveyors that move the gaming items in a rotational direction rather than a linear direction.

FIGS. 6A and 6B show an example of a game of chance coupled with an illumination source for sterilizing the user interface or area adjacent to the user interface.

FIG. 7 illustrates a gaming table having a user interface comprising the surface of the table.

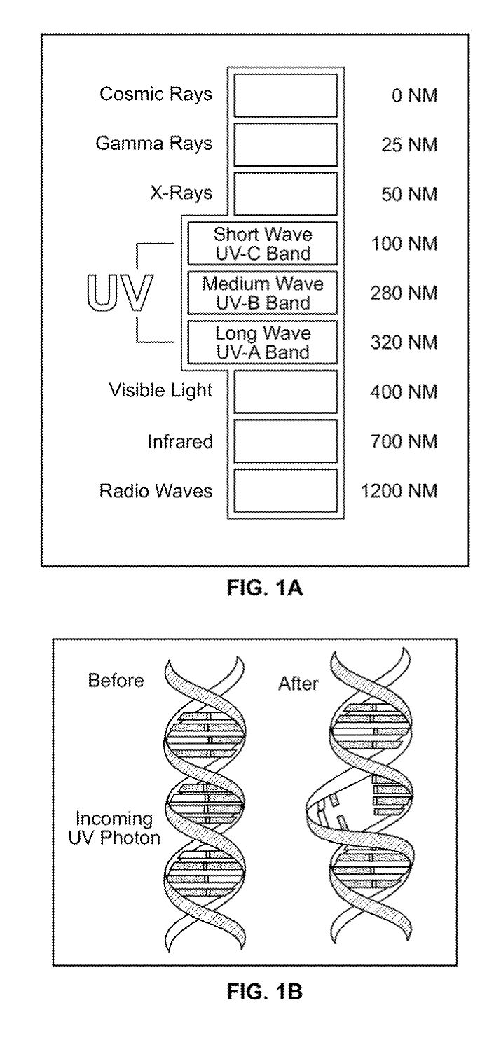

### DESCRIPTION OF THE INVENTION

One variation of the inventions described herein include a conveying system that allows for passing a number of items, such as casino chips, dice, cards, cash, coins, tokens, game cards (e.g., bingo cards, etc.) or other items commonly found in a casino through an enclosure for the purpose of sterilizing and/or sanitizing the item.

The systems of the present invention sterilize and/or sanitize the items based on a product of time and intensity—both levels must be specified for a successful sterilization. In one variation, the system delivers between 10,000-250,000 mWs/cm2 throughout the enclosure. The exposure will vary based on the application. For example, this exposure is believed to eliminate 95% to 99.9% of e.coli. Staphylococci, Streptococcus, Influenza, and Hepatitis. However, some pathogens may require a lesser dose. For example, Bacillus megatherium, dies at 2,500 units. Accordingly, the system may deliver any amount required to sterilize the target pathogen.

In other words, in the case of sterilization, the systems may incorporate sterilization means, as described herein, to eliminate pathogens. In the case of sanitization, the systems may incorporate additional means to clean and sanitize the items in addition or in place of sterilization before or after the sanitization process described herein.

While the conveyance system can be manual (e.g., such as a tray that is removable via an individual) ideally, the conveyance system will be automated so that it could run unassisted. Such systems are thought to be preferred for industries such as gaming where large amounts of items require sanitization but where it is desirable to minimize the number of staff that comes into contact with such items. For example, the inherent security precautions taken in the gaming industry require high throughput of the items where the processing is performed under guarded or secure conditions.

UV-A and UV-C: In one variation, the system uses a UV-C light based system alone. This configuration allows for quick sterilization, to a high degree, and without high temperatures (dry heat, steam), large pressure changes (ETO) or a wet process (Hypochlorites, like common household bleach or Ozone (O₃)). Currently, for gaming applications, it is presently believed that 253 nm to a dosage of 24,000 mW/s cm² is sufficient. However, use of UV-C light based systems is often preferred when the user is protected from the light. In those applications where protection is difficult, UV-A is preferred. The illumination sources of UV light can include UV generating lamps or light emitting diodes (LEDs).

In an additional variation, the systems may include UV plus a photocatalyst. One example of a photocatalyst is TiO2 (titania). However, other photocatalysts are also considered to be within the scope of variations of the system. The UV plus photocatalyst configuration provides the benefits of UV as well as an increase in the degree of surface sterilization (per time or watts of power consumed). One consideration with this system is that all game items (e.g., cards, dice, chips) must include a photocatalyst coating. Accordingly, items that were not manufactured with a photocatalyst or in which the photocatalyst degrades would not receive the degree of benefit from this procedure.

There are several techniques for applying a photo-catalyst or TiO2 to a surface for the purposes described herein. In one variation, the photo-catalyst can simply be added to the resin when the part consists of a molded part. In another variation, TiO2 can be applied in a coating process (either via a slurry or suspension). Similar such coatings are applied in the coatings of shower doors where the coating is applied in a visually transparent manner. For example, see Green Quest Technology Inc. Costa Mesa, Calif.

The properties of TiO2 are well known as agents for maintaining clean environments. For example, see Titanium-Oxide Photocatalyst, Three Bond Technical News 62, Jan. 1, 2004 and Photocatalytic Properties of Titania Complex under Visible Light Irradiation, Theories and Applications of Chem. Eng., 2005, Vol. 11, No. 1, the entireties of which are incorporated by reference herein. Such disclosures teach methods gel-sol methods of TiO2 coatings.

In another variation, the photo-catalyst can be coated via a number of processes known to those skilled in the art. (E.g., via a plasma spray, physical vapor deposition, cold plasma, or thermal spraying).

Ozone: Another variation for incorporation in the system include Ozone in Air (or nitrogen or other inert gas) and Ozone in water Solution (or alcohol/water) solution or acetic acid (vinegar)/water solution.

For the ozone in water solution, the items (may not be applicable for items such as cards) are fed into a wet hopper that leads to a tube or chamber filled with a solution with O₃ added at a concentration of 2.0 to 100.0 ppm. For those cases where the solution creates a risk of toxic byproduct, the system may be placed in a sealed environment or chamber.

A lead-oxide or tungsten cathode and an anode in the water solution can charge the solution with Ozone through a catalyst reaction (commonly understood by those skilled in the art of ozone generation). The solution will be largely based on purified water, but, may have a low concentration of acid like acetic acid or a low concentration of ethanol (alcohol) to speed the effectiveness of Ozone. The items are swirled or mixed to ensure even concentration of O₃ and even exposure for all pieces. The items spend a dosage period of up to 30 minutes in the tube or chamber. Next, the chips are removed for drying. Chips proceed to standard drying procedure (again, common to those skilled in the art, the important part being that you do not let water pool on the parts to avoid water spots or the few remaining pathogens to multiply).

For the ozone in air variations, items are fed into a dry hopper that leads to an opaque tube/chamber that is filled with a gas (all inert gases with some oxygen are OK). However, use of a flammable gas, such as pure oxygen is not typically desired as it presents the risk of an explosion. Moreover, high levels Of CO₂ have the potential of breaking down into poisonous CO (when the ozone creation strips it of an oxygen atom). O₃ is then added to the gas at a concentration of 0.05 ppm or less. If the chamber is designed not to let this gas out of the chamber (sealed, with recovery catalyst), even higher concentrations of O₃ can speed the process. However, in the interest of safety, it may be desirable to have a “safe to breathe” level of Ozone. Two tungsten electrodes will create Ozone in any gas mixture with some moisture and some oxygen in it (commonly understood by those skilled in the art of ozone generation). Clearly, regular air is the easiest gas to obtain, but, a nitrogen/oxygen/water vapor mixture could also work well.

The solution will be largely based on a gas, but, may have a low concentration of acid like acetic acid or a low concentration of ethanol (alcohol) to speed the effectiveness of Ozone. The game pieces are swirled or mixed with the gas/ozone mixture to ensure even exposure for all pieces. This could happen in an air hopper, much like the air popcorn poppers (literally a tube that blows parts straight up, they tumble, fall to the bottom and are blown up high again by the stream of air up the center). The chips spend a dosage period of up to 60 minutes in the tube or chamber. However, use of a high concentration permits a significant reduction in the dosage period (e.g., significantly less than 10 minutes. Next, the game pieces are removed for sorting and return to use. This has the advantage of being a dry process, but, it requires either a sealed chamber and a concentration of Ozone that is not safe to breathe (higher than 0.05 ppm according to the US EPA) or a long cycle time (e.g., ten minutes or longer).

UV plus Ozone: This variation is a wet process that where the item is fed into a wet hopper that leads to a dear tube (made of fused quartz or fused silica glass or other translucent material which does not absorb UV). The tube is filled with a solution as described above, with O3 added at a concentration of 2.0 to 4.0 ppm and/or if the UV contained some light below 180 nm (short wave UV-C) the UV light will generate O3 in the water solution

Higher concentrations of Ozone would speed the cycle lime but would require that the Ozone charged water be allowed a sealed space to “breathe” before disposal or another catalyst to remove Ozone before disposal. Game pieces are swirled or mixed in the water solution to ensure even concentration of O₃ and even exposure of UV-C for all game pieces. Outside the tube, UV lamps or LEDs are positioned such that they can shine 253 nm light on the item. Swirling jets may be provided inside the tube in order to introduce enough turbulence to expose all sides of the game pieces to UV. The items spend a dosage period of 1-20 minutes in the tube, receiving a dosage of 24,000 mW/s cm2 of U light during this period, then the chips are fed to standard continuous based drying. This process is takes longer than others because the tube requires a greater distance between the UV-C lamp and a game pieces; the sterilization rate decays with the cube (3rd power) of the distance from the UV light source.

UV plus Ozone Plus Photocatalyst: This variation is similar to that above with the addition of using a coating of photocatalyst on the item to assist in the sterilization process. As noted above, the photocatalyst speeds the cycle time for the same sterilization rate, or, increase the sterilization rate in the same cycle time.

Conveyance Means: The conveyance means illustrated can optionally include seats for placement of the items. For example, the seats can include feet or cleats (each track link) that can attach to standard conveyor chains and drives; this gives us the benefit of uniform spacing (which in turn provides uniform dosage). Additional features may be incorporated with the conveyance systems. For example, the conveyance system can have seats comprising recesses to accommodate the casino chips, cards or dice. Additional conveyance means include a drum (radial movement and radial mounted sanitization source). In such a case, the items would tumble within the drum for a sufficient time until sanitization occurs. In another variation, the conveying system may include a vibration table at a slightly declined angle or with air or water assist, or a tube filled with air or water that is made from fused quartz or fused silica glass and will thus allow UV light transmission. Presently, the illustrated conveyor systems are believed to provide the most even dosage of UV with the least custom-made parts.

The illustrated systems also demonstrate the need to sanitize each side of the item. For example, in the illustrated variation, two conveyance systems are used in series to flip or tumble the item. This variation allows for the use of sterilization source from one side of the item. Variations of the invention include exposing the item to sterilization sources from multiple sides so long as the conveying equipment does not significantly hinder the ability of the source to sterilize the items. For example, in one variation a clear vibration table made from a material that can transmit the sterilization source could be used. Of course, the intensity of the source may be required to be increased due to interference of the material. For example, a material that transmits 10% of the incident light may require a light source 10 times that of the unhindered light source.

It is noted that in variations of the invention-the conveyance systems should not deform or alter the items in any way. Such requirement is paramount in systems intended to clean gaming items where any mark or alteration of a chip, dice, or card could render that item unsuitable for use.

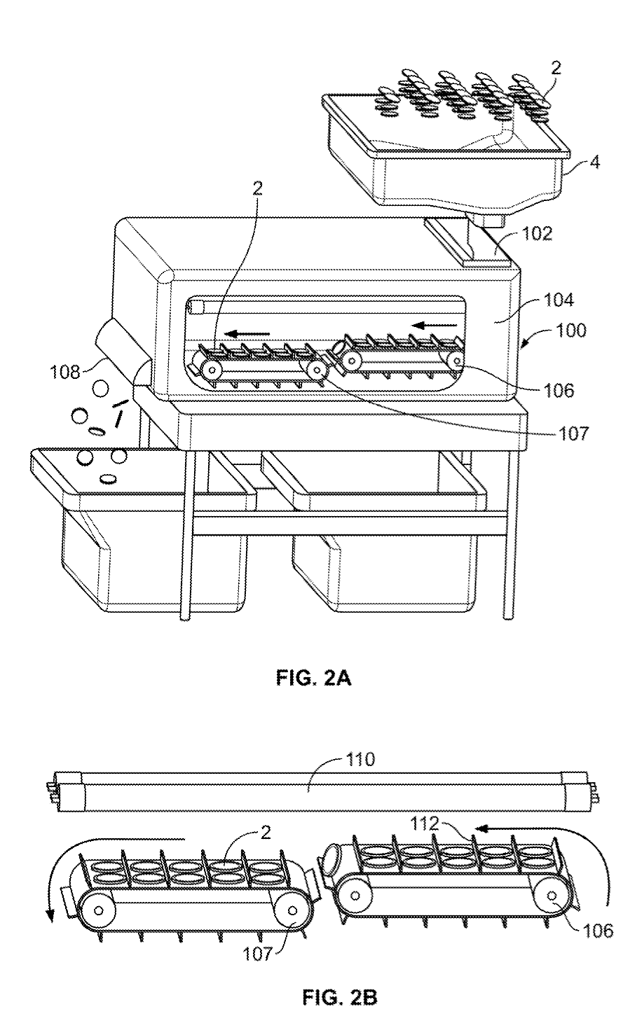

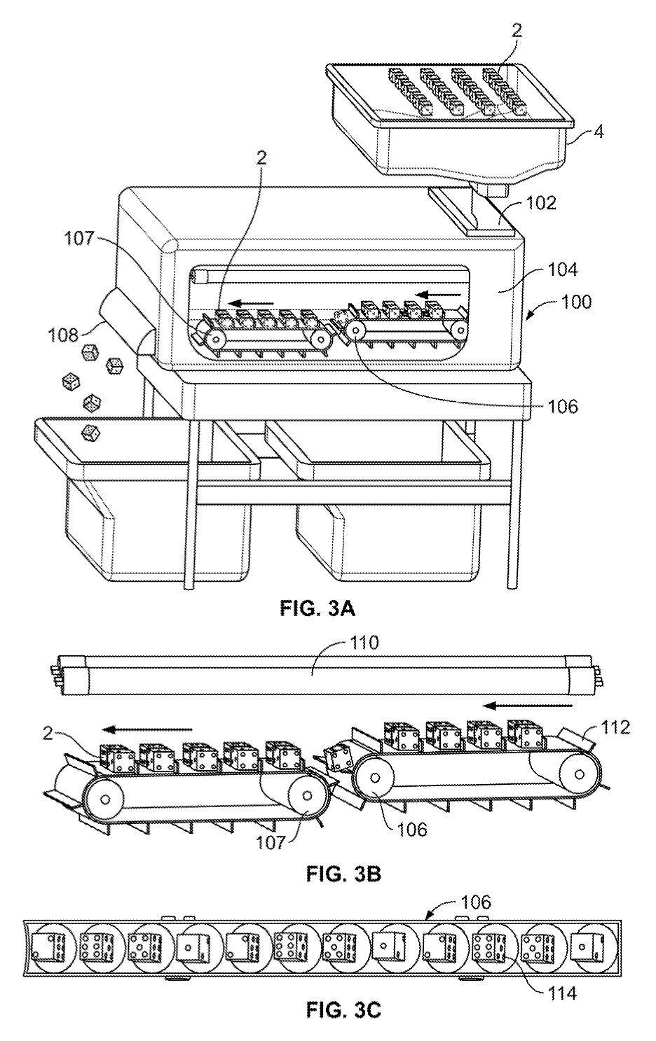

FIGS. 2A and 3A illustrate examples of systems 100 in accordance with the principles of the present invention. As shown, the system 100 may include an enclosure 104 having an opening for feeding the items 2 (in this case casino chips) into the system 100. As noted, the system 100 can be combined with any number of manual or automatic feeding units 4 as commonly known and used in manufacturing, assembly, and cleaning industries.

The enclosure 104 also includes an exit 108 from which the items 2 discharge from the unit 100 upon completion of the sterilization/sanitation cycle. While the illustration shows the items 2 being discharged into bins, variations of the invention include incorporating stacking or sorting equipment that organizes the items as they discharge.

Although additional variations are within the scope of this disclosure, the items 2 shown in the illustrated variation advance through the system 100 on a conveyor system. As shown the conveyor system can include a first conveyor portion 106 and a second conveyor portion 107. However, any number of conveyor portions are within the scope of the invention. In addition, some variations of the system may be used in circumstances where external observation of the sterilization process is desired for security or other purposes. In such a case, the system 100 can include a window 116 or other opening to allow visual observation of the sterilization process. In many cases, the window shall be designed to protect the observer from any harmful effects of the sterilization source. However, in some cases, the opening may simply be an opening in a wall of the enclosure. In addition, cameras or other remote imaging devices can be employed in lieu of or in addition to a window.

As noted above, the system 100 can be designed to sanitize/sterilize all sides of an item 2. In this variation, the system 100 comprises the two conveyor portions 106, 107 in an arrangement such that the transition between conveyor portions 106, 107 causes the item 2 to change in orientation or “flip” sides. It is noted that although the illustrations show casino chips, variations of the system may accommodate any of the items discussed herein.

In some variations of the invention, the conveyor system can be designed such that a light-source illuminates a surface of the element that is in contact with the conveying system. In such variations, the conveying system shall either be visually transparent to UV light or shall have sufficient porosity (e.g., windows, apertures, wires or a mesh-like configuration) to allow sterilization of the downward surface.

As shown in FIG. 2A, variations of the system include sterilization units 100 that provide a conveyor system 106, 107 that provides direct exposure of a surface of the item 2 to a sterilization source 110. As noted above, in those cases where the item is untreated, it will be important for the sterilization source 110 to directly expose a surface of the item to ensure sufficient sterilization of the item 2. Such a system allows for efficient sterilization. In contrast, if the unit included excessive rollers, excessive mechanisms for handling, or even excessive fluid, the ability to sterilize the element may be diminished. The ability to directly expose surfaces of the element to the sterilization source is also important when the element is treated with a coating or other photo-catalyst that aids in the sterilization process.

FIG. 2B illustrates internal components of the system 100 of FIG. 2A. As shown, this variation includes a sterilization source 110. The sterilization source 110 may be any of the UV sources described herein. Alternately, or in combination, the enclosure 104 may be configured to accommodate the fluids or gasses described in the additional sterilization/sanitation modes described above.

FIG. 2B also shows that the conveyor system may incorporate seats or separators 112 to space the units 2 across a desired distance. Such spacing is especially useful to prevent the items from overlapping that could otherwise interfere with the sterilization/sanitation process. The separators 112 can be selected based on the item being processed. For example, separators used for casino chips may have a different shape than separators for cards.

FIG. 2B also illustrates the UV source 110. In this variation, the UV source 110 comprises two lights that process multiple 1×2 array of items. However, other configurations are considered to be within the scope of this disclosure. For example, if a high output sanitization of items is required, the system may process any number of 1×N row of items with the required number of illumination sources to effect proper sanitization. Moreover, the number of items does not have to be related to the number of illumination sources 110 used. For example, in a current variation, the system sterilizes 12 items wide and uses 18 illumination sources.

As discussed above, the systems of the present invention sterilize and/or sanitize the items based on a product of time and intensity—both levels must be specified for a successful sterilization. The length of travel of the items 2 within the system 100 may be selected to provide the items to a sufficient exposure in view of the intensity of the sterilization source 110. In the illustrated variation, which uses dual conveyors for treatment of both sides of the item, the travel length is doubled so that each side of the item 2 receives sufficient exposure to the sterilization source 110.

FIGS. 3A to 3C illustrate another system 100 in accordance with the present invention. In this variation, the items 2 are illustrated as dice. However, much of this system is similar to the one shown in FIGS. 2A to 2B. FIG. 3C illustrates a variation where the conveyor system includes portions 106 and 107 where each portion has seats that comprise recesses 114 in the respective conveyor portion 106, 107 to accommodate the item being sterilized. Though not shown, the seats may comprise both separators 112 and recesses 114.

Although the above illustrations demonstrate the system as processing pairs of items, it is noted that the throughput of the system may vary as desired depending on the rate of sterilization required. For example, in some variations, the size of the enclosure or housing may be such that the unit is portable and can be placed adjacent to the gaming area. In such a case, the throughput of the portable device may be reduced.

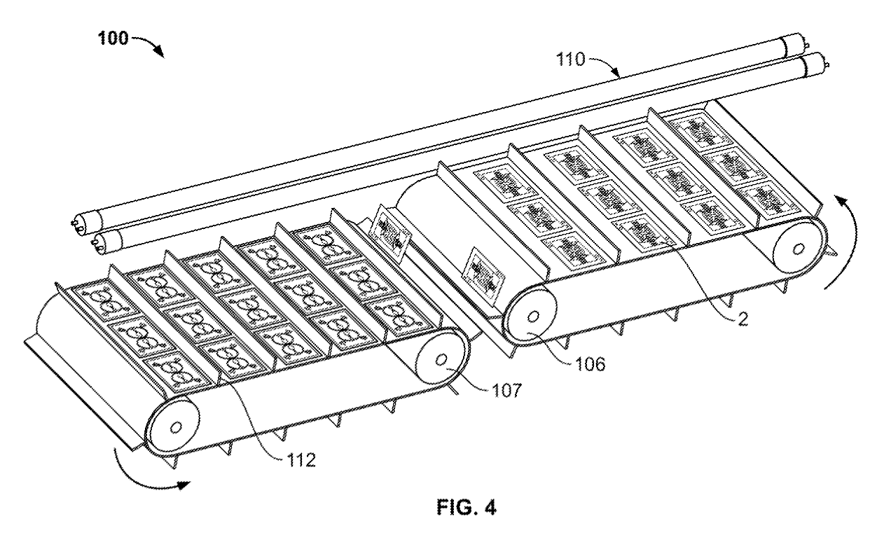

FIG. 4 illustrates another variation of a system according to the present invention. As shown, in this variation, the conveying system 106, 107 are configured to sterilize playing cards 2. As with the previous variations, the system 100 is designed to provide direct exposure of a surface of the card 2 on a first conveying portion 106 and direct exposure of a second surface of the card 2 on a second conveying portion 107. As noted above, the conveying portions 106 and 107 are arranged so that when the element 2 moves from the first portion to the second portion, the element is re-oriented to allow direct exposure of a surface of the card 2 that was previously obscured by the conveyor system.

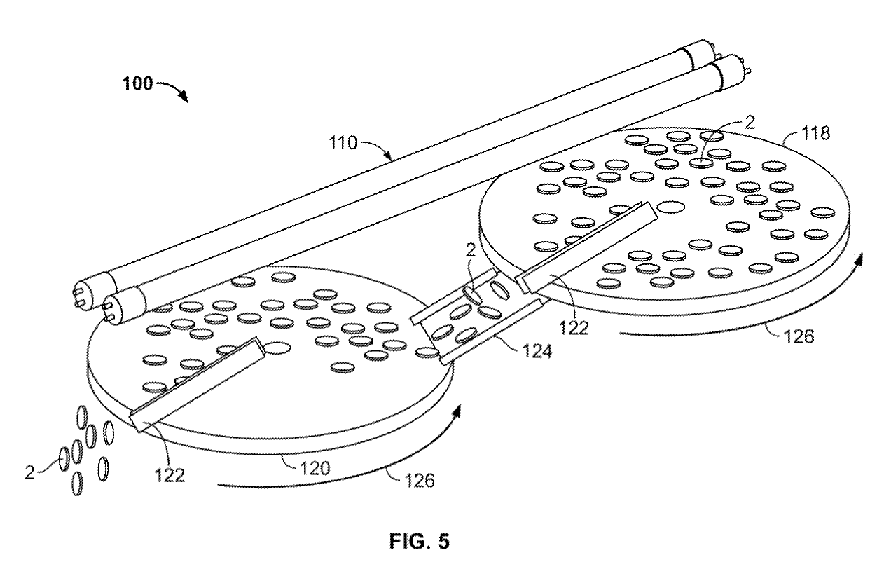

FIG. 5 illustrates another variation of a system 100 under the present invention. In this variation, the system 100 includes a conveying system comprising of a first conveyor portion 118 and a second conveying portion 120 where the conveying portions 118, 120 move the items 2 in a rotational direction rather than a linear direction as indicated by arrows 126. Although many of the principles of the present variation are similar to that of the variations described above, the use of a rotational conveyor system 118, 120 can increase the sterilization duty cycle time while minimizing a size of the unit.

As shown, the conveying portions 118, 120 allow for the sterilization unit 110 to directly expose a first surface of the element 2 (on conveyor 118). An optional transition element 124 can assist the elements 2 in moving to the second conveyor portion 120 in a manner in which the orientation of the element is different on the second conveyor portion 120. The end effect is that the sterilization element 110 is able to provide direct exposure to both sides the element. Clearly, any number of feed and removal mechanism 122 can be used to move the elements on or off of the conveyor system. While not shown, the conveying portions 118, 120 can include any number of separators, dividers, or recesses to accommodate the respective element.

As noted herein, the invention includes the systems described herein where the systems may have various combinations of the above described modes of sterilizing and sanitizing the item. Furthermore, the invention specifically includes systems in which the item 2 contains a photocatalyst or other substance that aids in reducing the amount of pathogens on the item after processing.

In addition to coating an item as described above with a photocatalyst, the invention described herein includes a system, such as a machine or apparatus with a user interface and an illumination source that illuminates over at least the user interface portion to sterilize and/or sanitize the user-interface.

For example, one variation of the invention includes a gaming machine or table (such as a slot machine, video poker machine, video blackjack machine, keno machine, card table, or similar game of chance type machine or table), where an illumination source is placed within a line-of-sight of the user interface (i.e., the portion that a user contacts to operate the machine or that a user rests on while operating the machine). The user interface may comprise button, levers used to actuate the machine, an electronic touch-display, the felt covering of a gaming table and/or sections of the machine/fixture where it is likely a user will rest their hands. Variations of the system may include shielding such that the illumination source does not expose the user to the illumination. In some variations, the illumination source and shielding may be retrofit onto the machine.

A first variation includes a system 200 where the user interface portion 202 of the machine or apparatus includes a photocatalyst to accomplish sterilization. The photocatalyst may be contained as an additive to a coating that is applied to the user interface. Alternatively, or in combination the photocatalyst may be an additive that is combined with the user interface or portions thereof during fabrication of the respective item. Typically, the additives are placed on the portions of the user interface that a user will contact when using the system. For example, as described herein, the user interface may include a photocatalyst. Upon exposure to the illumination source, the photocatalyst (e.g., TiO2—titanium dioxide) either directly sanitizes the user interface surface or improves the ability of the UV-C illumination source to sterilize the user interface. The photocatalyst may be applied as a film covering, or may be incorporated into the materials used to form the respective portion of the user interface. For example, in the gaming machine variation, a photocatalyst can be incorporated into a button, touch screen or lever to sterilize the surface.

In some cases, user interface may have pieces suited for frequent replacement as opposed to replacing the entire user interface. For example, such pieces may include buttons or other coverings that shield the electronics or actual switches of the equipment. Replacing these pieces allows for replenishing the photo-catalyst without replacing the entire user interface. It should be noted that the photocatalyst is not consumed by the sterilization reaction, however, scuffing and abrasion during use may require replenishment of the photocatalyst.

It should be noted that any UV source may be used in the various embodiments that are used without enclosures. However, a particular UV light source (e.g., UV-A, UV-B, or UV-C) may be preferred by consumers due to safety precautions.

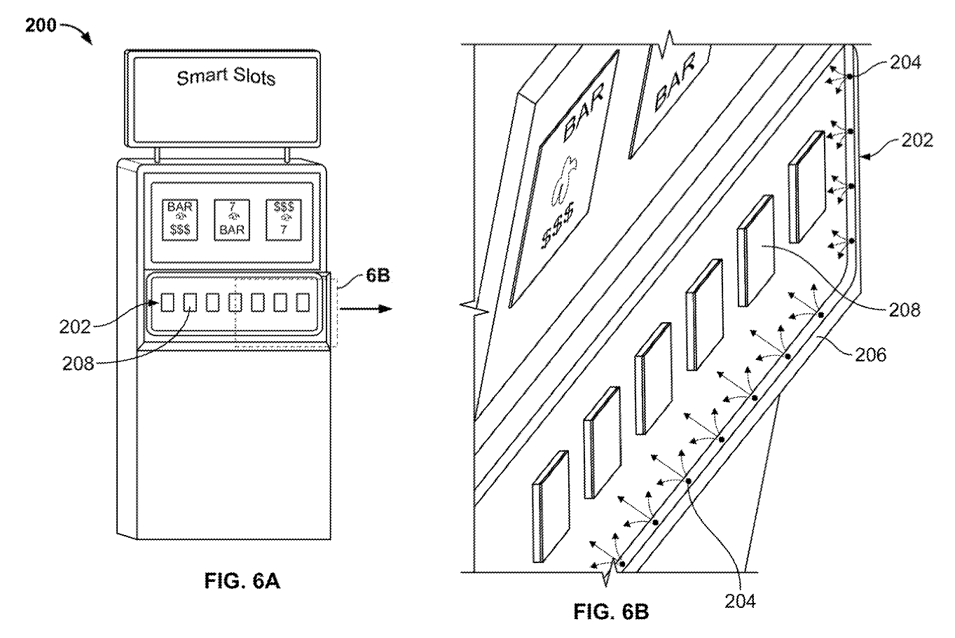

FIG. 6A illustrates an example of a system 200 in accordance with the principles of the present invention. As shown, the system 200 typically includes a user interface 202. In the illustrated example, the user interface 202 is the area operated by a user of a gaming machine. However, the user interface may comprise a touch-screen, keypad, lever, or any such feature that a user engages to operate the equipment.

FIG. 6B illustrates an expanded view of the section 6B from FIG. 6A. This figure illustrates an example of an illumination source 204 that is positioned to apply light based energy to the user interface 202. In this variation, the system 200 includes a plurality of illumination sources 204. However, variations of the system 200 may include one or more illumination sources, fixed or moveable over portions of the user interface 202.

As noted above, the illumination source may work in conjunction with a photo-catalyst to improve the ability of the system to sterilize. The photo-catalyst may be placed on the user interface as a film of material. Alternatively, buttons 208 or other removable components (that are frequently touched by the user) may be formed with a photocatalyst. In this manner, the portion of the user interface 202 having the photo-catalyst may be replaced without replacing the entire user interface 202. Variations of the invention also include fabrication of a photo-catalyst in a significant portion of the user interface 202 rather than in select components. For example, the entire region in the user interface 202 of FIG. 6A could be constructed with the photo-catalyst.

The arrangement of the illumination source and photo-catalyst may be placed within a number of areas on such equipment and is not limited to the example shown. Clearly, the illumination source and/or shall be placed to expose areas that are handled by users. For example, if the machine has a lever, the lever would be illuminated by the illumination source. Alternatively, the illumination source could be embedded within the actual lever. Moreover, in an alternate configuration, the illumination sources may be placed directly within the portion being handled. For instance, the illumination source may be placed underneath a surface of the user interface as long as a sufficient window exists so that the UV energy exposes the required user interface surface.

FIG. 6B also shows another optional aspect of the system 200 that protects the user from excessive exposure to the illumination source 204. As shown, a shield 206 can direct the illumination source 204 towards the user interface 102 and away from the user. For example, the illumination source 204 may be fully or partially covered by a perimeter bezel (much like a “C” section) that has a reflective interior and opaque outer surface. This configuration directs most of the UV light onto the user interface, rather than towards the users face or body. The cover or bezel may be fabricated in a manner such that it affixes to existing equipment.

The systems of the present invention may also be fabricated to prevent inadvertent exposure through a variety of sensing means. For example, the illumination source 204 may be designed to trigger only during certain hours where it is unlikely that a user operates the equipment. In other cases, the illumination source and associated control system may be coupled to a sensing means (such as a motion detector or a sensing circuit that detects activity in the system). In this manner, if a user is detected through movement or system activity, the illumination source may be terminated or operate at a lower intensity.

In another aspect of the system, the user interface may be constructed with a photo-catalyst but no illumination source. Such a system could be sterilized in areas having sufficient ambient UV-A light (produced to a limited degree by standard commercial indoor light) or sunlight.

Additionally, LED UV-A lamps mounted in a bezel around the perimeter of the play area may accelerate the sterilization rate. With a set of standard parts, a small quantity of titania and about 1 hour time, a service technician can take a standard slot machine or video poker and convert it to a self-cleaning system.

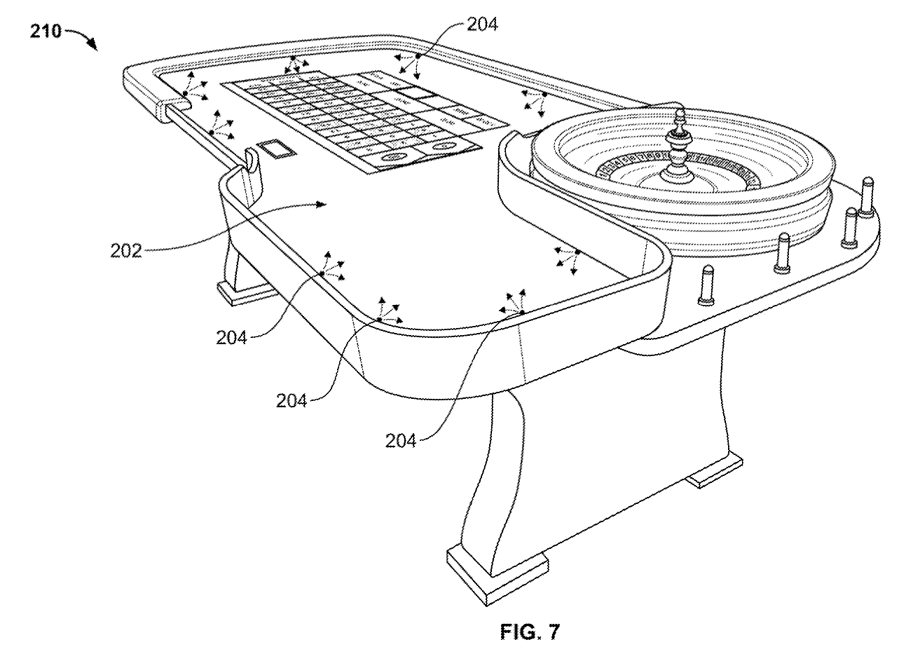

FIG. 7 illustrates another variation of a gaming table 210 under the present invention. As shown, the gaming table 210 comprises a user surface or interface 202 that is primarily covered by a felt or other similar material. The material is treated with a photo-catalyst so that either ambient illumination or illumination by optionally placed illumination sources 204 sterilize the user interface 202.

As noted herein, the invention includes the systems described herein where the systems may have various combinations of the above described modes of sterilizing and sanitizing the item. Furthermore, the sterilizing means may include retrofit kits (comprising either replacement parts having the photocatalyst and/or illumination sources) that may be installed on existing devices and machines that would benefit from the sterilization process discussed herein.

The invention further includes various methods of processing such items to reduce the number of pathogens or substances on the item. Such methods include the use of such items (e.g., casino chips, dice or cards) that incorporate an additional photocatalyst.

We claim:

1. A sterilizing unit for sterilizing gaming items comprising:
a housing;
a light generating sterilizing source;
a conveyor system having at least a first conveyor portion and a second conveyor portion, each conveyor portion placed such that a plurality of surfaces of the gaming item are directly exposed to the light generating sterilizing source; and
where the first conveyor portion includes a first plurality of seats for nesting of at least one of the gaming items, and where the second conveyor portion includes a second plurality of seats for nesting at least one of the gaming items, where the first and second conveyor portions are placed relative to one another such that movement of the gaming item from the first conveyor portion to the second conveyor portion causes the gaming item to change orientation when nested in the second conveyor portion.

2. The sterilizing unit of claim 1, where the sterilizing source comprises a UV light source located within the housing and coupled to a power supply such that the light source is configured to generate light between 100 and 400 nanometers wavelength.

3. The sterilizing unit of claim 2, where the sterilizing source provides 100,000-250,000 mWs/cm2 throughout the housing.

4. The sterilizing unit of claim 1, where the sterilizing source comprises a source of ozone in a gas state.

5. The sterilizing unit of claim 1, where the housing is portable.

6. The sterilizing unit of claim 1, where the first and second plurality of seats comprises at least one raised member attached to the respective conveyor portion.

7. The sterilizing unit of claim 1, where the first and second plurality of seats comprises at least one recess in the respective conveyor portion.

8. The sterilizing unit of claim 1, where at least one of the first and second conveyor portions comprise a linear conveyor surface.

9. The sterilizing unit of claim 1, where at least one of the first and second conveyor portions comprise a rotational conveyor surface.

10. The sterilizing unit of claim 1, where at least one of the first and second conveyor portions comprise a vibrational table.

11. The sterilizing unit of claim 1, where the housing includes a visually transparent section allowing for external viewing of the gaming items within the housing.

12. A sterilizing unit for sterilizing gaming items comprising:
a housing;
a sterilizing means;
a conveyor system having at least a first conveyor portion and a second conveyor portion, each conveyor portion placed such that the gaming item is exposed to the sterilizing source; and
where the first conveyor portion includes a first plurality of seats for nesting of at least one of the gaming items, and where the second conveyor portion includes a second plurality of seats for nesting at least one of the gaming items, where the first and second conveyor portions are placed relative to one another such that movement of the gaming item from the first conveyor portion to the second conveyor portion causes the gaming item to change orientation when nested in the second conveyor portion.

13. The sterilizing unit of claim 12, where the sterilizing means comprises a sterilizing source selected from the group consisting of UV light, gas ozone, and a combination thereof.

14. The sterilizing unit of claim 12, where the housing is portable.

15. The sterilizing unit of claim 12, where the first and second plurality of seats comprises at least one raised member attached to the respective conveyor portion.

16. The sterilizing unit of claim 12, where the first and second plurality of seats comprises at least one recess in the respective conveyor portion.

17. The sterilizing unit of claim 12, where at least one of the first and second conveyor portions comprise a linear conveyor surface.

18. The sterilizing unit of claim 12, where at least one of the first and second conveyor portions comprise a rotational conveyor surface.

19. The sterilizing unit of claim 12, where at least one of the first and second conveyor portions comprise a vibrational table.

20. The sterilizing unit of claim 12, where the housing includes a visually transparent section allowing for external viewing of the gaming items within the housing.

21. A method for providing a gaming item sterilization system, the method comprising:
creating a sterilization path having a light-based sterilization source within an enclosure;
advancing the gaming item on a conveyor through a portion of the sterilization path, such that at a plurality of surfaces of the gaming item are directly exposed to the sterilization source advancement through the sterilization path; and
directly exposing each surface of the gaming item to the light-based sterilization source for a sufficient time to significantly sterilize all surfaces of the gaming item without damaging the gaming item.

22. The method of claim 21, where directly exposing each surface of the light-based sterilization source to the light-based sterilization source comprises automatically re-orienting the gaming item so that all surfaces of the gaming item are exposed to the light-based sterilization source.

23. The method of claim 21, where the gaming item further includes a circuitry and wherein exposing the light-based sterilization source to each side of the gaming item to significantly sterilize the surface of the gaming item without damaging the circuitry.

24. The method of claim 21, further comprising applying a photocatalyst surface onto a portion of the surfaces of the gaming item such that the photocatalyst reacts with the light-based sterilization source to sterilize the surfaces of the gaming item.

25. The method of claim 21, where the sterilizing source comprises a UV light source located within the enclosure and is coupled to a power supply such that the UV light source is configured to generate light between 100 and 400 nanometers.

26. The method of claim 25, where the sterilizing source provides 10,000-250,000 mWs/cm2 throughout the housing.

27. The method of claim 21, where the enclosure is portable and further comprising re-locating the enclosure on a gaming floor.

28. The method of claim 21, where advancing the gaming item on the conveyor comprises at least advancing the gaming item on a first conveyor portion and a second conveyor portion where the conveyor portions are located such that transfer of the gaming item from the first conveyor portion to the second conveyor portion causes reorientation of the gaming item when on the second conveyor portion.

29. The method of claim 21, where the gaming item comprise an item selected from the group consisting of a casino chip, a coin. a die, and a playing card.

30. A self-sterilizing casino gaming machine for engaging a user in a casino game, the machine comprising:
a display portion for visually displaying the game; and
a user interface comprising one or more portions allowing the user to engage in the game and affect the display portion, where at least a portion of the user interface comprises a photo-catalyst material that when irradiated with UV light, sterilizes the portion of the user interface.

31. The machine of claim 30, where the photo-catalyst material comprises TiO2.

32. The machine of claim 30, where the portion of the user interface comprises a structure selected from a group consisting of a button, a touch screen, and a lever.

33. The machine of claim 30, further comprising at least one illumination source placed within a line-of-sight of the portion of the user interface.

34. The machine of claim 33, where the illumination source comprises a UV light source coupled to a power supply such that the light source is configured to generate light between 100 and 400 nanometers wavelength.

35. The machine of claim 33, where the illumination source is located behind a covering, such that the covering provides at least a partial shield to a user from the illumination source.

36. A self-sterilizing casino gaming table comprising:
a playing surface having at least a covering portion containing visual marks of a casino game; and
a photo-catalyst material located on at least a surface of the covering portion without obscuring the visual marks of the casino game, where when irradiated with UV light, the photo-catalyst material sterilizes the covering portion.

37. The casino gaming table 36, where the photo-catalyst material comprises TiO2.

38. The casino gaming table 36, where the covering portion comprises a felt material.

39. The casino gaming table 36, further comprising at least one illumination source placed within a line-of-sight of the surface of the covering material.

40. The casino gaming table 39, where the illumination source comprises a UV light source coupled to a power supply such that the light source is configured to generate light between 100 and 400 nanometers wavelength.

41. The casino gaming table 39, where the illumination source is located behind a covering, such that the covering prevents an individual from being irradiated by the illumination source.
